(19) United States
(12) Patent Application Publication
(10) Pub. No.: US 2014/0020331 A1
(43) Pub. Date: Jan. 23, 2014

(54) Microwave sterilizing device for containers

(71) Applicants: KUANG-TSE CHIN, Taichung City (TW); HUNG-NAN TUNG, Taoyuan City (TW); JUNG-KUEI HSIEH, Caotun Township (TW)

(72) Inventors: KUANG-TSE CHIN, Taichung City (TW); HUNG-NAN TUNG, Taoyuan City (TW); JUNG-KUEI HSIEH, Caotun Township (TW)

(73) Assignee: CAN PACK COMMERCIAL CO., LTD

(21) Appl. No.: 13/662,558

(22) Filed: Oct. 29, 2012

(30) Foreign Application Priority Data
Jul. 18, 2012 (TW) 101213811

(51) Int. Cl. A61L 2/12; A61L 2/18; B65B 55/04

(52) U.S. Cl. 53/167; 422/304

(57) ABSTRACT

A microwave sterilizing device includes a container supplying device, a container transport device, a sterilizing sprayer, a microwave cavity unit and a microwave generating device. The container supplying device may supply containers in a continuous manner. The container transport device has a starting point and an end point and the containers are moved from the starting point to the end point on the container transport device. The container supplying device is disposed near the starting point. The sterilizing sprayer can spray germicide onto the inside and outside of each container. The microwave cavity unit can allow the passage of the containers. The microwave generating device is disposed outside of the microwave cavity unit and can generate microwave which is fed into the microwave cavity unit to heat up the germicide remained on the internal walls of each container so as to sterilize the container.

### BACKGROUND OF THE INVENTION

1. Field of the invention

The invention generally relates to a sterilizing device for containers. More particularly, the invention relates to a sterilizing device in which microwave is used to heat up germicide so as to sterilize containers swiftly.

2. Description of the prior art

As of now, sterilizing devices have been commonly used when moving content into containers in the food and beverage packaging process. Different types of sterilizing methods are used for different types of packaging materials, including paper, plastic material and laminated material. Currently, to optimize the sterilizing efficiency, chemical germicides have been used. Most commonly used chemical germicides have been PAA (peracetic acid) and hydrogen peroxide. In practice, the peroxide is heated up to its boiling point to transform it into a gaseous state which is then mixed with air. Then the gaseous mixer is sprayed onto each container to kill germs and microorganisms. Then, sterile water is used to rinse out the containers. Finally, the containers are let dry in germ-free air.

Such wet type sterilizing method has been conventionally used. However, because germicide is often discomposed into water and oxygen and because of the larger amount of waste water needs to be collected and treated as well other problems, such as the wet condition of the content filling chamber, excess amounts of germicide and sterile water need to be used and the fact that foul smell may be generated, as of now, dry type sterilizing method by the use of peroxide has become more popular.

In the realm of dry type sterilizing method, the PET container electron beam sterilizing and filling machine developed by Mitsubishi Heavy Industries Food& Machinery Co. has the advantages of eco-friendliness, energy saving, low water consumption and free of chemical germicide. PET containers and their caps are sterilized by “air rinse” and electron beams. The report of the ISBT (the International Society of Beverage Technologists) 2010 describes the benefits of the use of electron beams which may reduce “food miles” by 80 to 90% and reduce water consumption.

Microwave heats up objects containing polar molecules (such as water in the liquid form) by the oscillation of these polar molecules. When an object exposes to microwave radiation (typically with a frequency of 2.4 G Hz), its polar molecules (especially water molecules) would oscillate along the oscillating electric field and would have translational movement which in turn causes the rise of temperature. Though microwave radiation may cause non-polar molecules to have a slight amount of movement and polarization, such movement and polarization contributes virtually nothing to the rise of temperature. Therefore, if an object contains several types of materials, these materials would react differently to microwave radiation because they have different dielectric constants.

Therefore, there is a need to develop a container sterilizing device that is energy saving, energy efficient, effective and safe.

### SUMMARY OF THE INVENTION

A microwave sterilizing device for containers is disclosed. The microwave sterilizing device of the present invention comprises a container supplying device, a container transport device, a sterilizing sprayer, a microwave cavity unit and a microwave generating device. The container supplying device may supply containers in a continuous manner. The container transport device has a starting point and an end point and the containers are moved from the starting point to the end point on the container transport device. The container supplying device is disposed near the starting point. The sterilizing sprayer can spray germicide onto the inside and outside of each container. The microwave cavity unit can allow the passage of the containers. The microwave generating device is disposed outside of the microwave cavity unit and can generate microwave which is fed into the microwave cavity unit to heat up the germicide remained on the internal walls of each container so as to sterilize the container. These components are arranged in the order of the container supplying device, sterilizing sprayer, microwave cavity unit and microwave generating device from the starting point to the end point.

In an embodiment of the present invention, the microwave sterilizing device further comprises a content filling device which may move the content into each sterilized container.

In another embodiment of the present invention, the microwave sterilizing device further comprises a control device which can control the container transport device, sterilizing sprayer, microwave cavity unit and microwave generating device.

In addition, the microwave sterilizing device further comprises an electricity supplying device which may be electrically connected with the container transport device, sterilizing sprayer, microwave cavity unit and microwave generating device.

In yet another embodiment of the present invention, the microwave sterilizing device further comprises a plastic container forming machine.

In yet another embodiment of the present invention, the container transport device includes a conveyor belt. The sterilizing sprayer has a nozzle, which may stick into each container.

The containers may be made of various types of materials that may be microwaved, such as paper, glass, pottery, porcelain, wood or a plastic material.

### BRIEF DESCRIPTION OF THE DRAWINGS

The features and purposes of the microwave sterilizing device of the present invention would be better understood if the following is read with the accompanied drawings.

FIG. 1 is a side view schematically illustrating the microwave sterilizing device of the present invention, with lighter lines which represent a plastic container forming machine, and darker lines which represent the components of the microwave sterilizing device.

FIG. 2 is a side view schematically illustrating the microwave cavity unit and the microwave generating device of the microwave sterilizing device of the present invention.

### DETAILED DESCRIPTION OF THE PREFERRED EMBODIMENT

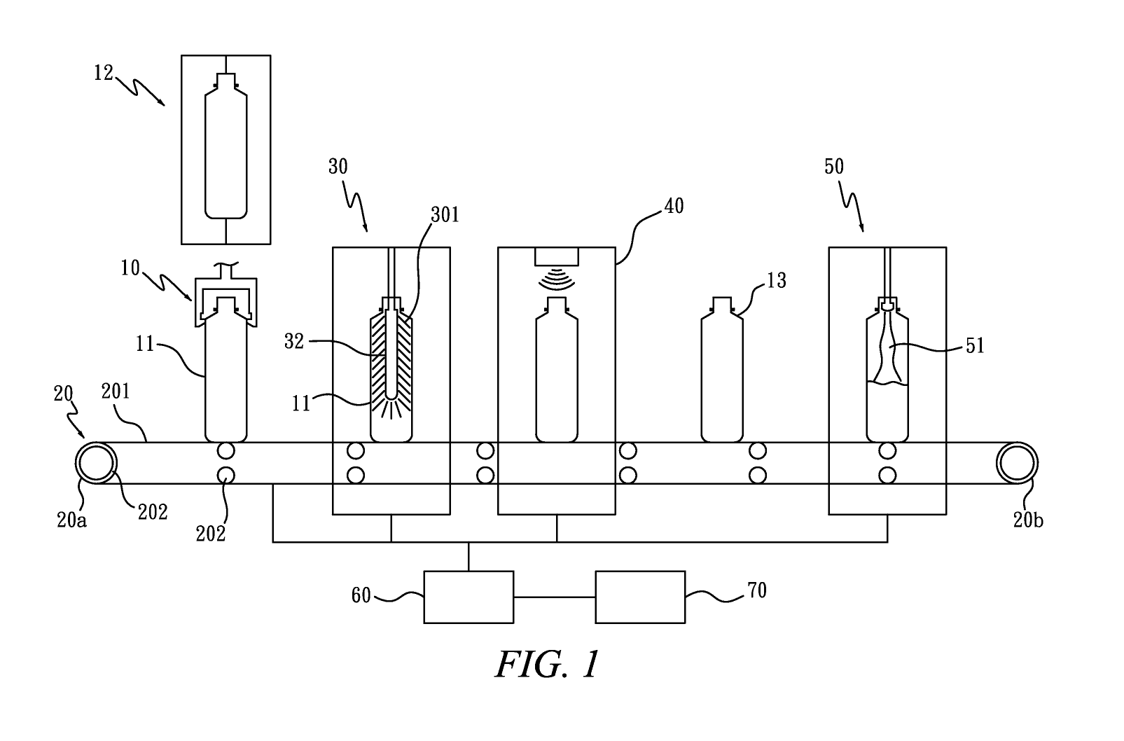

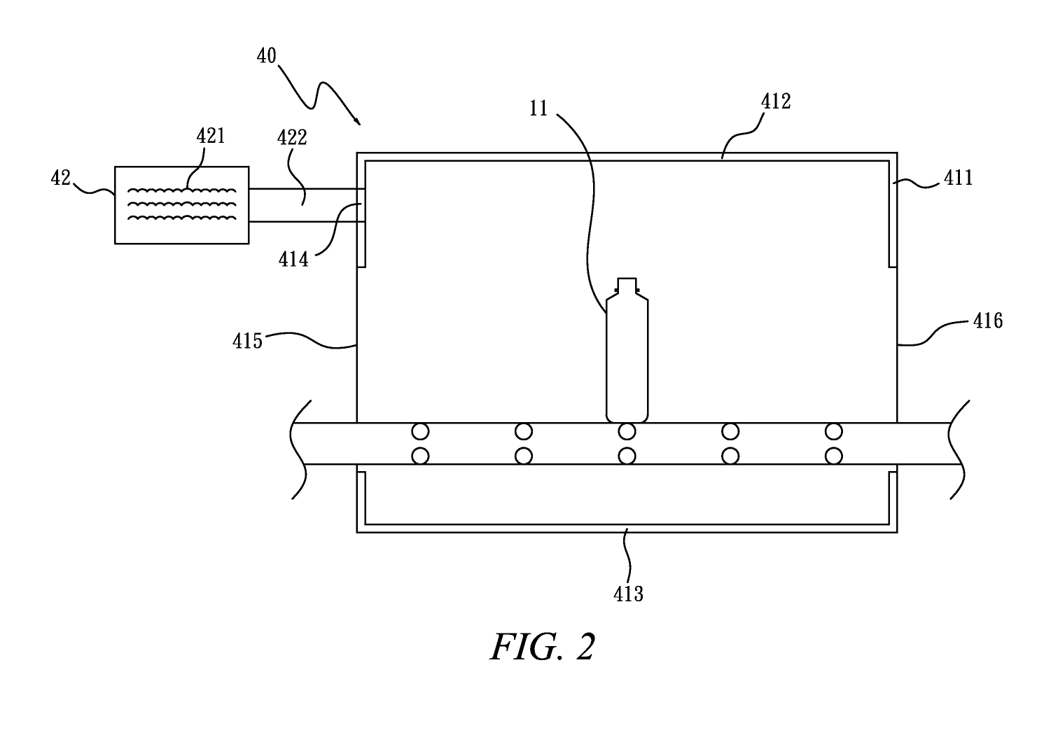

The features and purposes of the microwave sterilizing device of the present invention would be better understood if the following is read with the accompanied drawings (FIGS. 1 and 2).

Please see FIGS. 1 and 2, which illustrate the microwave sterilizing device of the present invention. The microwave sterilizing device of the present invention comprises a container supplying device 10, a container transport device 20, a sterilizing sprayer 30, a microwave cavity unit 40 and a microwave generating device 42. Also, these components are arranged in the order of the container supplying device 10, sterilizing sprayer 30, microwave cavity unit 40 and microwave generating device 42 from the starting point 20a to the end point 20b.

The microwave sterilizing device of the present invention may be used to sterilize containers made of various types of materials, such as paper, glass, pottery, porcelain, wood and plastic materials by heat. In the preferred embodiment of the present invention, plastic containers (preferably, a container made of polyester) are sterilized.

The container supplying device 10 is able to supply containers 11 in a continuous manner. The container supplying device 10 may be a device that can move the plastic containers made by a forming machine 12 to a holding portion, which may be a fixture, of the container transport device 20.

The container transport device 20 may consist of a conveyor belt 201 and several rollers 202. The container transport device 20 has a starting point 20a and an end point 20b. Containers 11 are moved from the starting point 20a to the end point 20b on the container transport device 20. The container supplying device 10 is disposed near the starting point 20a. The container transport device 20 may be a holding device or other type of device.

The sterilizing sprayer 30 may spray the disinfectant or germicide 301 onto the inside and outside of each container 11. The sterilizing sprayer 30 has a nozzle 32 which may stick into each container 11. In the preferred embodiment of the present invention, the germicide 301 is peroxide or hypochlorite (sodium hypochlorite), preferably, hydrogen peroxide and peracetic acid. Then, after the container 11 is sprayed by the germicide 301, it is moved by the container transport device 20 to the microwave cavity unit 40.

The microwave cavity unit 40 has dimensions in the range from 5 to 30 cm. If the microwave cavity unit 40 is a rectangular cube, its dimensions are preferably 20×20×20 cm. A microwave generator 42 is disposed outside of the microwave cavity unit 40 and can generate microwave and feed it into the microwave cavity unit 40 so that the germicide 301 retained on the inner walls of the container 11 may absorb the microwave and then heat up to further fulfill its disinfecting function. Afterwards, a sterilized container 13 may be obtained.

As illustrated in FIG. 2, the microwave cavity unit 40 has two side walls 411, an upper wall 412 and a lower wall. A window 414 that allows the entry of microwave into the microwave cavity unit 40 is provided in one of the side walls 411. The lower portion of either side wall 411 has an opening 415 and 416 to allow the entry and exit of each container 11 transported by or riding on the container transport device 20.

A microwave generator 42 is disposed outside of the microwave cavity unit 40 and comprises a magnetron 421 and a waveguide 422. The magnetron 421 can generate microwave and the waveguide 422 connects magnetron 421 with the microwave cavity unit 40 through a window 414. Microwave generated by the magnetron 421 is fed to the microwave cavity unit 40 through the waveguide 422 and the window 414. Because the window 414 is disposed in a side wall 411, no additional interfering device is needed. Also, microwave may be fed from any direction, not limited to the side.

Containers 11 enter the microwave cavity unit 40 through the opening 415. Then, microwave generated by the microwave generator 42 is fed to the microwave cavity unit 40 through the window 414 and the microwave heats up the container 11 quickly. The microwave also swiftly heats up the germicide 301 evenly sprayed on the inner walls of the container 11 to its boiling point so as to make the germicide 301 evaporate and thus turn the container 11 into a sterilized container 13. In the preferred embodiment of the present invention, the microwave generated by the microwave generator 42 has a frequency from 2205 to 2695 MHz (such as 2450 MHz) or from 823 to 1007 MHz (such as 915 MHz) and the heating time of each container 11 is from 5 to 120 seconds.

Therefore, not only the germicide 301 can kill bacteria and microorganisms, but the high temperatures caused by the heat-up of the germicide 301 by the microwave also have a sterilizing function. Because the containers may be made of various types of materials, which may have different dielectric constants (or relative permittivities) (please see Table 1) higher than that of the germicide, the containers would not be heated up as quickly as the germicide. In the preferred embodiment of the present invention, the containers are made of a plastic material, the containers would not be heated up as quickly as the germicide to avoid deformation.

The microwave sterilizing device of the present invention also includes a content filling device 50 which may move the content 51 into the sterilized containers 13.

The microwave sterilizing device of the present invention may also include a control device 60, which can control the container transport device 20, sterilizing sprayer 30, microwave cavity unit 40 and microwave generating device 42 and even the container supplying device 10 and content filling device 50.

The microwave sterilizing device of the present invention may also include an electricity supplying device 70 which may be electrically connected with the control device 60, container transport device 20, sterilizing sprayer 30, microwave cavity unit 40 and microwave generating device 42.

TABLE 1
Dielectric constants (or relative permittivities) for different types
of germicides and containers:

| Material | Dielectric constant |
|---|---|
| Water | 80 |
| Alcohol | 16-31 |
| Hydrogen peroxide | 84.2 |
| Bleaching powder | 4.5 |
| Polystylene | 2.55 |
| Paper | 2.7 |
| Glass | 5.05 |
| Wood | 1.82 |
| Pottery | 5.51 |
| Raw rubber | 2.05 |
| Nylon | 2.84 |
| PET | 3.37 |
| PP | 2.3 |
| HDPE | 2.35 |

Method 1: to determine microorganisms remaining on the inside of a container
Necessary items and method:
Necessary items:
NB culture, microwave oven, water bath tank, electronic probe type thermometer and timer

### Method:

Pour 600 ppm of the water solution of peracetic acid into a first container made of polyester. Pour 900 μL of deionized water into a second container made of polyester (used as a comparison). Then, add 100 μL of water solution with bacillus subtilis spores into each container to make the total amount of fluid in each container to reach 1 mL. Next, place the first container in a 1000 W microwave oven for 10 to 20 seconds and is cooled down in the water bath tank. Then, measure the number of microorganisms.
Method 2: to determine the amount of residual germicide remaining on the inside of a container

Pour 1800 ppm of the water solution of peracetic acid into a container made of polyester.

Then, place the container in a 1000 W microwave oven to heat up the container. Next, use 5 mL of deionized water to rinse out the inside of the container. The rinsed out water is poured into a flask and then add 45 mL of deionized water into the flask. Next, use 0.1 N potassium permanganate for titration so as to determine the amount of residual peracetic acid.

By these methods, we conclude that, in terms of sterilization or germ killing, microwave can kill germs in a shorter time than the conventional heating method. Also, the combination of the use of microwave as the heating method and germicide, sterilization may be accomplished in a shorter time (the amount of bacillus subtilis spores may be reduced substantially). By the use of such combination, germicide with a lower concentration may be used. Therefore, the amount of residual germicide remaining on the container may be greatly reduced (to 0.5 ppm). Through these two methods, it has been proven that the microwave sterilizing device of the present invention is energy saving, energy efficient, effective and safe.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A microwave sterilizing device for containers, comprising:
a container supplying device, able to supply containers in a continuous manner;
a container transport device, including a conveyor belt and several rollers, and having a starting point and an end point, wherein containers are moved from the starting point to the end point on the container transport device and the container supplying device is disposed near the starting point;
a sterilizing sprayer, able to spray germicide onto the inside and outside of each container;
a microwave cavity unit, allowing the passage of the containers; and
a microwave generating device, disposed outside of the microwave cavity unit and able to generate microwave which is fed into the microwave cavity unit to heat up the germicide remained on the internal walls of each container so as to sterilize the container, wherein the container supplying device, sterilizing sprayer, microwave cavity unit and microwave generating device are arranged in order from the starting point to the end point.

2. The microwave sterilizing device as in claim 1, further comprising a content filling device which may move the content into each sterilized container.

3. The microwave sterilizing device as in claim 1, further comprising a control device which can control the container transport device, sterilizing sprayer, microwave cavity unit and microwave generating device.

4. The microwave sterilizing device as in claim 1, further comprising an electricity supplying device which may be electrically connected with the container transport device, sterilizing sprayer, microwave cavity unit and microwave generating device.

5. The microwave sterilizing device as in claim 1, further comprising a plastic container forming machine.

6. The microwave sterilizing device as in claim 1, wherein the germicide is peroxide.

7. The microwave sterilizing device as in claim 6, wherein is hydrogen peroxide or peracetic acid.

8. The microwave sterilizing device as in claim 1, wherein the germicide is hypochlorite or sodium hypochlorite.

9. The microwave sterilizing device as in claim 1, wherein the microwave generated by the microwave generator has a frequency from 2205 to 2695 MHz.

10. The microwave sterilizing device as in claim 1, wherein the microwave generated by the microwave generator has a frequency from 823 to 1007 MHz.

11. The microwave sterilizing device as in claim 1, wherein a heating time of each container by microwave is from 5 to 120 seconds.

12. The microwave sterilizing device as in claim 1, wherein the containers may be made of as paper, glass, pottery, porcelain, wood or plastic material.

13. The microwave sterilizing device as in claim 1, wherein the container transport device includes a conveyor belt.

14. The microwave sterilizing device as in claim 1, wherein the sterilizing sprayer has a nozzle which may stick into each container.

15. The microwave sterilizing device as in claim 1, wherein the microwave cavity unit has dimensions of length, width and height) in the range from 5 to 30 cm.
